(19) United States
(12) Patent Application Publication
(10) Pub. No.: US 2021/0233635 A1
(43) Pub. Date: Jul. 29, 2021

(54) INDIVIDUALIZED DIALYSIS WITH INLINE SENSOR

(71) Applicant: Fresenius Medical Care Holdings, Inc., Waltham, MA (US)

(72) Inventors: Shashikant Dattatraya Kalaskar, Layton, UT (US); Vivian Changna Wang, Farmington, UT (US)

(21) Appl. No.: 17/156,224

(22) Filed: Jan. 22, 2021

Related U.S. Application Data
(63) Continuation-in-part of application No. 16/811,533, filed on Mar. 6, 2020.
(60) Provisional application No. 62/967,349, filed on Jan. 29, 2020.

(51) Int. Cl. G16H 20/10; A61M 1/16; G06K 9/62; G16H 10/60

(52) U.S. Cl. CPC G16H 20/10; A61M 1/1603; A61M 2205/35; G06K 9/6218; G16H 10/60; G06K 9/6276

(57) ABSTRACT

A system for determining individualized dialysis prescriptions is provided. The system comprises a prescription recommendation server and an on-demand dialysis machine. The prescription recommendation server is configured to: receive, from a prescriber computing device, patient information associated with a new patient; determine, based on the patient information, an individualized dialysis prescription for the new patient, wherein the individualized dialysis prescription indicates a particular patient cluster associated with the new patient; and transmit, to an on-demand dialysis machine, the individualized dialysis prescription for the new patient. The on-demand dialysis machine is configured to: receive, from the prescription recommendation server, the individualized dialysis prescription for the new patient; and perform a dialysis treatment on the new patient based on the individualized dialysis prescription.

### CROSS-REFERENCE TO RELATED APPLICATIONS

This patent application is a continuation-in-part of and claims the benefit of U.S. patent application Ser. No. 16/811,533, filed on Mar. 6, 2020. This patent application also claims the benefit of U.S. Provisional Patent Application No. 62/967,349, filed Jan. 29, 2020. Both of which are incorporated by reference herein in their entirety.

### BACKGROUND

Patients with kidney failure or partial kidney failure typically undergo hemodialysis treatment at hemodialysis treatment centers, clinics, or in the home. When healthy, kidneys maintain the body's internal equilibrium of water and minerals (e.g., sodium, potassium, chloride, calcium, phosphorous, magnesium, and sulfate). In hemodialysis, blood is taken from a patient through an intake needle (or catheter) which draws blood from a vein located in a specific access location (arm, thigh, subclavian, etc.). The blood is then pumped through extracorporeal tubing via a peristaltic or other pump, and then through a special filter called a dialyzer. The blood passes through the dialyzer in contact with an internal semipermeable membrane, typically in a countercurrent direction to the flow of a dialysate solution on the opposite side of the membrane. The dialyzer is intended to remove unwanted toxins such as urea, creatinine and exchange essential electrolytes like potassium and/or sodium. Further, the dialyzer is intended to remove excess water from the blood by diffusion and./or convective transport, depending on the specific type of dialysis ordered. The dialyzed blood then flows out of the dialyzer via additional tubing and through a needle (or catheter) back into the patient.

During dialysis, an excess of electrolytes in the patient's blood may be lost. Also in some cases, dialysis may result in insufficient removal of electrolytes. For example, blood contains sodium ions (Na⁺), potassium ions (K⁺), and calcium ions (Ca²⁺). Too much sodium in the blood can contribute to the patient feeling an increase in thirst or can lead to hypertension. Losing too much sodium can lead to decline in blood volume, chest pain, nausea, vomiting, headache, and muscle cramps. Too much potassium in the blood can lead to muscle pain, weakness, and numbness. Losing too much potassium can lead to heart rhythm disturbances. Having too much calcium in the blood can lead to vascular calcification. Losing too much calcium can lead to bone disorders and/or uncontrollable secondary parathyroid hormone (PTH) secretion.

Electrolyte composition in the blood is a highly dynamic function, dependent on many physiological and nutritional inputs, and subject to significant variability between patients. In most dialysis settings, one or only a small number of dialysate compositions (i.e., “recipes”) is available to treat patients, regardless of individual variations in electrolyte profiles that exist between patients or even between the same patient on different days. This “one-size-fits-all” approach to treatment may be reasonable for the majority of patients, but some patients do not tolerate it well. Accordingly, a method and system for preparing a patient-specific dialysate would be advantageous, and one that can adapt to real-time changes in patient needs between and even during dialysis treatments.

### SUMMARY

In an exemplary embodiment, the present application provides an individualized and on-demand dialysis system for determining individualized dialysis prescriptions, The system comprises a prescription recommendation server and an on-demand dialysis machine, The prescription recommendation server is configured to: receive, from a prescriber computing device, patient information associated with a new patient; determine, based on the patient information, an individualized dialysis prescription for the new patient, wherein the individualized dialysis prescription indicates a particular patient cluster associated with the new patient, and transmit, to an on-demand dialysis machine, the individualized dialysis prescription for the new patient. The on-demand dialysis machine is configured to: receive, from the prescription recommendation server, the individualized dialysis prescription for the new patient and perform a dialysis treatment on the new patient based on the individualized dialysis prescription.

In some instances, the prescription recommendation server is configured to determine the individualized dialysis prescription for the new patient based on using one or more dialysis prescription machine learning and/or artificial intelligence (AI-ML) models.

In some examples, the prescription recommendation server is configured to determine the individualized dialysis prescription based on using the one or more dialysis prescription AI-ML models by: inputting the patient information into the one or more dialysis prescription AI-ML models to determine the particular patient cluster, wherein the particular patient cluster is associated with a medical condition of the new patient, and determining the individualized dialysis prescription based on the particular patient cluster.

In some variations, the prescription recommendation server is further configured to: train the one or more dialysis prescription AI-ML, models based on received training information to determine associations within the received training information.

In some instances, the prescription recommendation server is further configured to: receive the training information, wherein the training information comprises past prescriptions provided to a plurality of patients, outcomes associated with performing dialysis treatment using the past prescriptions, and a plurality of recommended dialysis prescriptions.

In some examples, the one or more dialysis prescription AI-ML models comprises a supervised model. The supervised AI-ML model is a support vector machine (SVM) model or a K Nearest Neighbor (kNN) model.

In some variations, the prescriber computing device and the on-demand dialysis machine are both physically located at a prescriber's office.

In some instances, the prescriber computing device is physically located at a prescriber's office associated with a first geographical location, and the on-demand dialysis machine is physically located at a residence of the new patient. The residence is associated with a second geographical location that is different from the first geographical location,

In some examples, the prescription recommendation server is further configured to: transmit, to the prescriber computing device, the individualized dialysis prescription for the new patient; receive, from the prescriber computing device, prescriber information indicating one or more adjustments to the individualized dialysis prescription. The prescription recommendation server is configured to transmit the individualized dialysis prescription for the new patient by transmitting the individualized dialysis prescription with the one or more adjustments indicated by the prescriber information.

In another exemplary embodiment, the present application provides a method for determining individualized dialysis prescriptions. The method comprises: receiving, by a prescription recommendation server and from a. prescriber computing device, patient information associated with a new patient; determining, based on the patient information, an individualized dialysis prescription for the new patient, wherein the individualized dialysis prescription indicates a particular patient cluster associated with the new patient; and transmitting, to an on-demand dialysis machine, the individualized dialysis prescription for the new patient. The on-demand dialysis machine performs a dialysis treatment on the new patient based on the individualized dialysis prescription.

In some instances, determining the individualized dialysis prescription for the new patient is based on using one or more dialysis prescription machine learning and/or artificial intelligence (AI-ML) models.

In some examples, the method further comprises: inputting the patient information into the one or more dialysis prescription AI-ML models to determine the particular patient cluster, wherein the particular patient cluster is associated with a medical condition of the new patient; and determining the individualized dialysis prescription based on the particular patient cluster.

In some variations, the method further comprises: training the one or more dialysis prescription AI-ML models based on received training information to determine associations within the received training information.

In some instances, the method further comprises: receiving the training information, wherein the training information comprises past prescriptions provided to a plurality of patients, outcomes associated with performing dialysis treatment using the past prescriptions, and a plurality of recommended dialysis prescriptions.

In some examples, the one or more dialysis prescription AI-ML models comprises a supervised AI-ML model, The supervised AI-ML model is a support vector machine (SVM) model or a K Nearest Neighbor (kNN) model.

In some variations, the prescriber computing device and the on-demand dialysis machine are both physically located at a prescriber's office.

In some instances, the prescriber computing device is physically located at a prescriber's office associated with a first geographical location and the on-demand dialysis machine is physically located at a residence of the new patient. The residence is associated with a second geographical location that is different from the first geographical location.

In some examples, the method further comprises: transmitting, to the prescriber computing device, the individualized dialysis prescription for the new patient; and receiving, from the prescriber computing device, prescriber information indicating one or more adjustments to the individualized dialysis prescription. The method further comprises transmitting the individualized dialysis prescription with the one or more adjustments indicated by the prescriber information.

In yet another exemplary embodiment, a non-transitory computer-readable medium having processor-executable instructions stored thereon is provided. The processor-executable instructions, when executed, facilitate: receiving, by a prescription recommendation server and from a prescriber computing device, patient information associated. with a new patient; determining, based on the patient information, an individualized dialysis prescription for the new patient, wherein the individualized dialysis prescription indicates a particular patient cluster associated with the new patient; and transmitting, to an on-demand dialysis machine, the individualized dialysis prescription for the new patient. The on-demand dialysis machine performs a dialysis treatment on the new patient based on the individualized dialysis prescription.

In some instances, determining the individualized dialysis prescription for the new patient is based on using one or more dialysis prescription machine learning, and/or artificial intelligence (AI-ML) models.

### BRIEF DESCRIPTION OF THE DRAWINGS

FIG. 1 illustrates a front perspective view of a hemodialysis system that includes an electrolyte composition monitor according to some embodiments of the disclosure;

FIG. 2 is a block diagram illustrating use of an electrolyte composition monitor with a patient, according to embodiments of the disclosure;

FIG. 3 is a flow diagram for managing electrolytes in blood of a dialysis patient during dialysis, according to an embodiment of the disclosure;

FIG. 4 is an example timeline for managing electrolytes in blood of a dialysis patient during dialysis, according to an embodiment of the disclosure;

FIG. 5 is a flow diagram for determining individualized dialysate recipes or prescriptions for a patient;

FIG. 6 is a block diagram of an example computer system;

FIG. 7 shows graphical representations of real-time electrolyte concentration measurements using an NMR sensor;

FIG. 8 is a simplified block diagram depicting an exemplary individualized and on-demand dialysis system with network capabilities in accordance with an exemplary embodiment of the present application; and

FIG. 9 illustrates an exemplary process for using machine learning to provide individualized dialysis prescriptions and treatments to patients.

### DETAILED DESCRIPTION

During dialysis, an electrolyte composition monitor according to embodiments of the disclosure can employ a dialysate mixing system to make an amount of dialysate on demand using, among other things, a plurality of chemical concentrates. The dialysate generated will have a formula, recipe, or prescription that differs from dialysate previously used during dialysis. The dialysate's formula will thus be adjusted during dialysis based on the electrolyte composition monitor detecting an elevated or depressed level of one or more electrolytes in a patient's blood during dialysis.

In an embodiment, the dialysate used during dialysis is made in batches. Each batch follows a prescription, formula, or recipe chosen by the electrolyte composition monitor based. on receiving electrolyte concentration levels from the patient's blood. The electrolyte composition monitor may continually adjust a next dialysate batch's recipe and task its dialysate mixing system to follow the prescribed recipe. For example, the dialysate mixing system may receive a recipe indicating particular chemical constituents and amounts of each chemical constituent to be included in the dialysate. Based on the prescription, the dialysate mixing system can determine, for example, a number of tablets, mass of powder, or volume of concentrated electrolyte solution required for each chemical constituent. Tablets, powders and/or concentrated electrolyte solutions, can be automatically dispensed and mixed with purified water, bicarbonate, and/or sodium chloride in a mixing chamber to produce the dialysate according to the desired dialysate recipe.

Embodiments of the disclosure allow for chemical constituents to be delivered and stored in a tablet form or in a concentrated form, thus requiring minimal storage space and oversight. Mixing the dialysate in batches throughout dialysis suggests less storage space is required since the volume of dialysate made can be fully exhausted during a treatment session.

Embodiments of the disclosure allow for the dialysate composition used during dialysis to be personalized, whereby a patient's individual responses to dialysis are taken into account by monitoring his electrolyte responses to the dialysis treatment throughout the treatment session. In this way, a one-size-fits-all rule or a coarse heuristic is not applied during the treatment. The electrolyte composition monitor, through its continuous adjustments of dialysate composition, can effectively personalize treatment to the individual patient, ensuring that the patient does not leave the dialysis treatment with deficient levels or elevated levels of certain monitored electrolytes, and improving long-term outcomes and patient satisfaction.

Embodiments of the disclosure allow for an electrolyte composition monitor that can, over time, learn a dialysis recipe or formula most appropriate for the patient. By continually adjusting the dialysate in batches, the electrolyte composition monitor can determine which electrolytes the patient is typically sensitive to; thus, in further treatments, the electrolyte composition monitor can suggest a starting dialysate recipe that is more appropriate for the patient. In this way, embodiments of the electrolyte composition monitor will allow for a learning model tailored to adapt to evolving patient needs.

Embodiments of the disclosure provide individualized dialysis treatment based on online monitoring of electrolytes in the patient's blood by generating individualized dialysate as electrolyte conditions in the patient's blood change during treatment. This improvement solves a problem in current treatment practice where dialysate formulas and recipes for individual patients are based on monthly lab blood test results. Dialysis patients are rarely in steady state, so a lab blood test may be outdated by the time the patient enters the clinic for dialysis. Thus, reliance on monthly lab testing may prove harmful or of limited benefit to individual patients.

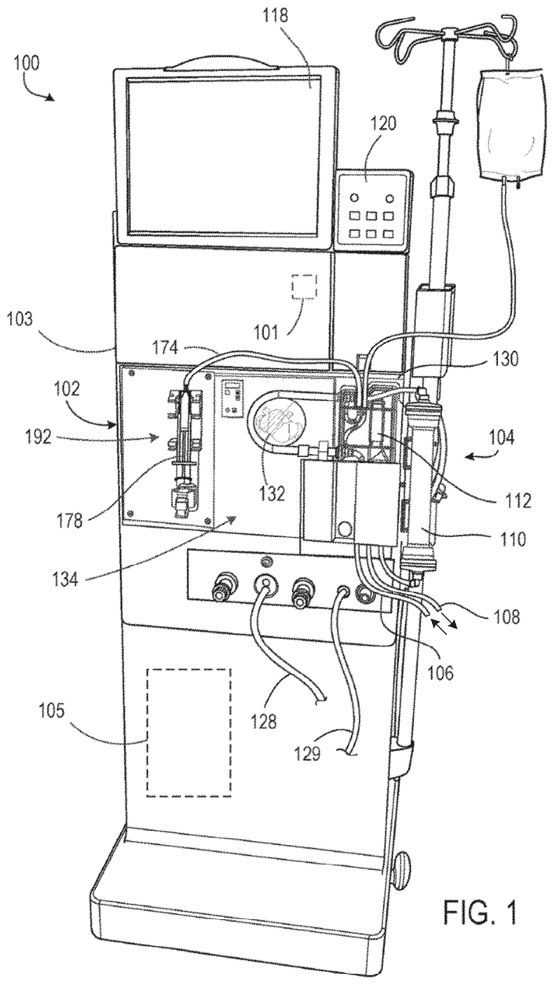

FIG. 1 shows a dialysis system, in particular, a hemodialysis system 100. Although the system described herein is largely described in connection with hemodialysis systems by way of example, it is explicitly noted that the system described herein may be used in connection with other types of medical devices and treatments, including peritoneal dialysis systems. The hemodialysis system 100 includes a hemodialysis machine 102 connected to a disposable blood component set 104 that partially forms a blood circuit. During hemodialysis treatment, an operator connects an arterial patient line 106 and a venous patient line 108 of the blood component set 104 to a patient. The blood component set 104 includes an air release device 112. As a result, if blood passing through the blood circuit during treatment contains air, the air release device 112 will vent the air to atmosphere.

The blood component set 104 is secured to a module 130 attached to the front of the hemodialysis machine 102. The module 130 includes a blood pump 132 capable of circulating blood through the blood circuit. The module 130 also includes various other instruments and sensors, e.g., electrolyte sensors, capable of monitoring the blood flowing through the blood circuit. The module 130 includes a door that when closed, as shown in FIG. 1, cooperates with the front face of the module 130 to form a compartment that is sized and shaped to receive the blood component set 104.

The blood pump 132 is part of a blood pump module 134. The blood pump module 134 includes a display window, a start/stop key, an up key, a down key, a level adjust key, and an arterial pressure port. The display window displays the blood flow rate setting during blood pump operation. The start/stop key starts and stops the blood pump 132. The up and down keys increase and decrease the speed of the blood pump 132. The level adjust key raises a level of fluid in an arterial drip chamber.

The hemodialysis machine 102 further includes a dialysate circuit formed by the dialyzer 110, various other dialysate components, and dialysate lines connected to the hemodialysis machine 102. Many of these dialysate components and dialysate lines are inside the housing 103 of the hemodialysis machine 102 and are thus not visible in FIG. 1. During treatment, while the blood pump 132 circulates blood through the blood circuit, dialysate pumps (not shown) circulate dialysate through the dialysate circuit.

The dialysate is created by the hemodialysis machine 102 in batches. That is, the hemodialysis machine 102 is configured to mix various chemical constituents of the dialysate together to form a dialysate batch having requisite characteristics based on measurements of electrolyte concentration in the patient's blood. In this way, dialysate used during the dialysis treatment can be optimized for the specific patient for different phases of the treatment based on how the patient is responding to the treatment.

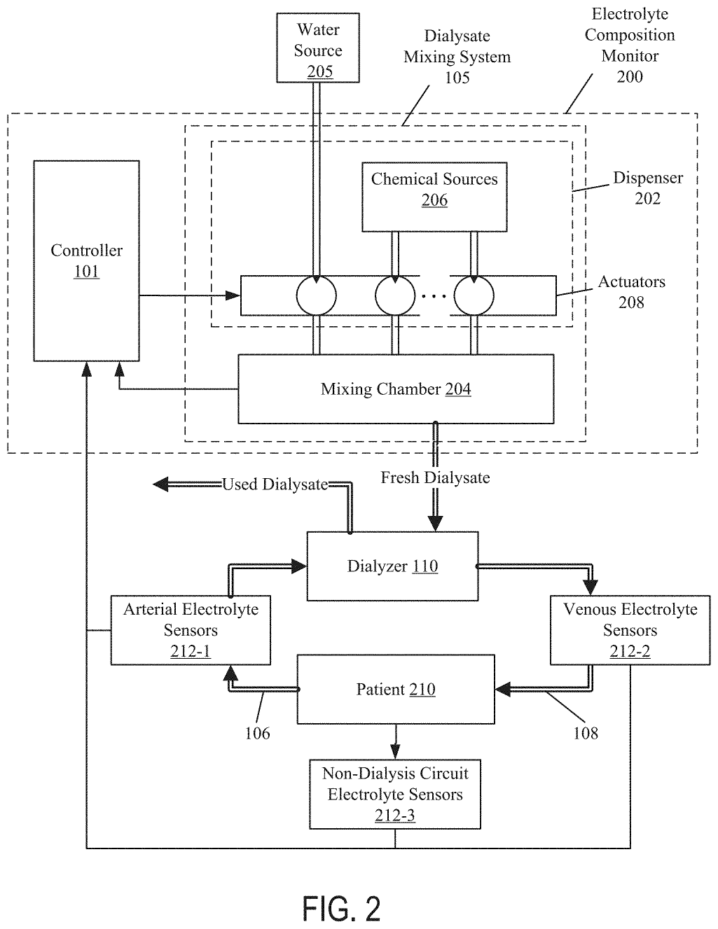

The hemodialysis machine 102 includes an electrolyte composition monitor (200 of FIG. 2), which is made up of a controller 101 and a dialysate mixing system 105 for mixing dialysate. During dialysis, the controller 101 is configured to receive electrolyte measurements from the patient's blood, and the controller 101 is configured to provide signals for adjusting the dialysate recipe for dialysate batches throughout the dialysis treatment, The dialysate mixing system 105 is internal to the housing 103 of the hemodialysis machine 102. In an embodiment, water, sodium chloride (NaCl), bicarbonate (NaHCO3), and a plurality of chemical concentrates are mixed together to form the dialysate. The dialysate mixing system 105 provides already mixed dialysate to the dialyzer 110 via at least a dialysate supply line, which is also internal to the housing 103 of the hemodialysis machine 102. A drain line 128 and an ultrafiltration line 129 extend from the hemodialysis machine 102. The drain line 128 and the ultrafiltration line 129 are fluidly connected to the various dialysate components and dialysate lines inside the housing 103 of the hemodialysis machine 102 that form part of the dialysate circuit. During hemodialysis, the dialysate supply line carries fresh dialysate through various dialysate components, including the dialyzer 110. As the dialysate passes through the dialyzer 110, it collects toxins from the patient's blood. The resulting spent dialysate is carried from the dialysate circuit to a drain via the drain line 128. When ultrafiltration is performed during treatment, a combination of spent dialysate and excess fluid drawn from the patient is carried to the drain via the ultrafiltration line 129.

In an embodiment, the controller 101 determines the chemical composition of each batch of dialysate. For example, a batch of dialysate may be 12 liters (L) and the chemical composition may include a plurality of chemical concentrates. The chemical concentrates may be liquid concentrates of varying viscosity and/or may be solid concentrates in the form of tablets, pills, or powders. The controller 101 may compute the chemical composition (e.g., an amount of each of the plurality of chemical concentrates such as a number of tablets) for each 12 L batch of dialysate based on a prescription issued by a physician/doctor.

In an embodiment, the controller 101 may use a reduced volume of the dialysate for the dialysis treatment of the patient. For example, the controller 101 may reduce the dialysate to blood flow ratio for the dialysis treatment. By reducing the dialysate to blood flow ratio, the dialysis treatment may consume less dialysate (e.g., 40 L of the dialysate per dialysis treatment may be used instead of 120 L).

A drug pump 192 also extends from the front of the hemodialysis machine 102. The drug pump 192 is a syringe pump that includes a clamping mechanism configured to retain a syringe 178 of the blood component set 104. The drug pump 192 includes a stepper motor configured to move the plunger of the syringe 178 along the axis of the syringe 178. The drug pump 192 can thus be used to inject a liquid drug (e.g., heparin) from the syringe 178 into the blood circuit via a drug delivery line 174 during use, or to draw liquid from the blood circuit into the syringe 178 via the drug delivery line 174 during use.

The hemodialysis machine 102 includes a user interface with input devices such as a touch screen 118 and a control panel 120. The touch screen 118 and the control panel 120 allow an operator to input various different treatment parameters to the hemodialysis machine 102 and to otherwise control the hemodialysis machine 102. The touch screen 118 allows an operator to select between user profiles, and the control panel 120 can allow the operator to select between user profiles by scanning the patient's membership card. The touch screen 118 displays information to the operator of the hemodialysis system 100. The controller 101 is also configured to receive and transmit signals to the touch screen 118 and the control panel 120. The controller 101 can control operating parameters of the hemodialysis machine 102, e.g., providing signals at appropriate times for adjusting composition of dialysate throughout a dialysis treatment. The dialysate mixing system can be, e.g., the dialysate mixing system in Kalaskar et al., US 2018/0326138, which is hereby incorporated herein in its entirety.

FIG. 2 is a block diagram illustrating use of an electrolyte composition monitor 200 with a patient 210 during dialysis, according to embodiments of the disclosure. Components of the hemodialysis system 100 of FIG. 1 are used as an example, but as previously stated, the electrolyte composition monitor 200 can be used in peritoneal dialysis. The electrolyte composition monitor 200 is configured to receive electrolyte measurements from one or more electrolyte sensors 212. The electrolyte composition monitor 200 is also configured to use the electrolyte measurements to adjust dialysate recipe, mix a new batch of dialysate, and provide fresh dialysate to the dialyzer 110.

The electrolyte composition monitor 200 includes the controller 101 and the dialysate mixing system 105. The controller 101 is configured to interface with the electrolyte sensors 212 to receive the electrolyte measurements. Examples of the controller 101 include a field programmable gate array (FPGA), an application specific integrated circuit (ASIC), and a processor with non-transitory computer-readable medium.

The dialysate mixing system 105 includes a dispenser 202 and a mixing chamber 204. The dispenser 202 can include chemical concentrates in chemical sources 206. The chemical concentrates are used as ingredients of the dialysate mixture. The chemical concentrates can be liquid concentrates of varying viscosity or can be solid concentrates in the form of tablets, pills, or powders. The chemical sources 206 are containers that hold these chemical concentrates. Chemical sources 206 can thus hold concentrates of potassium chloride (KCl), calcium chloride (CaCl₂), magnesium chloride (MgCl₂), citric acid, dextrose, sodium chloride (NaCl), sodium bicarbonate (NaHCO3), acetic acid, glucose, and so on. Not all chemical concentrates available need to be used in a dialysate formula or recipe. For example, one recipe may only call for acetic acid, NaCl, CaCl₂, KCl, MgCl₂, and glucose. Another recipe may call for bicarbonate, NaCl, CaCl₂, KCl, and MgCl₂, without glucose. Regardless, the dialysis composition should meet the intended prescription for a given patient and meet biocompatibility standards (e.g., pH of the dialysate).

The dispenser 202 can further include actuators 208 that aid in dispensing specific amounts of the chemical concentrates to a mixing chamber 204 for mixing a batch of dialysate. The actuators 208 not only control the amount of chemical concentrates provided to the mixing chamber 204, but also control an amount of water used in mixing the batch of dialysate. A water source 205 can be a water connection for receiving filtered water or water suitable for use in dialysis treatment. The water source 205 can connect to the hemodialysis system 100 via an inlet tube.

The dispenser 202 provides the chemical concentrates and water to the mixing chamber 204. Contents in the mixing chamber 204 are agitated for an appropriate amount of time until chemical concentrates are sufficiently distributed throughout, In sonic embodiments, the mixing chamber 204 increases its temperature to assist in dissolving and/or distributing the chemical concentrates to yield a homogenous solution. After realizing a homogenous solution, the mixing chamber 204 can be brought to an appropriate temperature for dialysis treatment.

The mixing chamber 204 of the dialysate mixing system 105 provides the mixed dialysate to the dialyzer 110. In an embodiment, the mixing chamber 204 is multi-chambered where a first chamber is used for mixing dialysate and a second chamber is used for storing and delivering dialysate to the dialyzer 110. In an embodiment, the mixing chamber 204 includes sensors for sensing levels of dialysate in both the first and second chambers. The mixing chamber 204 may also alert the controller 101 when a batch of dialysate has been mixed and when fresh dialyzer from the batch of dialysate is provided to the dialyzer 110.

The dialyzer 110 receives blood from the patient 210 via the arterial patient line 106. Electrolyte sensors along the arterial patient line 106 may be provided for measuring electrolyte concentration in blood upstream of the dialyzer 110. These electrolyte sensors are identified as arterial electrolyte sensors 212-1 in FIG. 2. The dialyzer 110 returns blood to the patient 210 via the venous patient line 108. Electrolyte sensors along the venous patient line 108 may be provided for measuring electrolyte concentration in blood downstream of the dialyzer 110. These electrolyte sensors can be identified as venous electrolyte sensors 212-2. Furthermore, electrolyte sensors not interrupting the dialysis circuit can be interfaced with the patient 210. For example, a sensor can be placed along a peripherally inserted central catheter (PICC) line to measure electrolyte concentration. These sensors are identified as non-dialysis circuit electrolyte sensors 212-3.

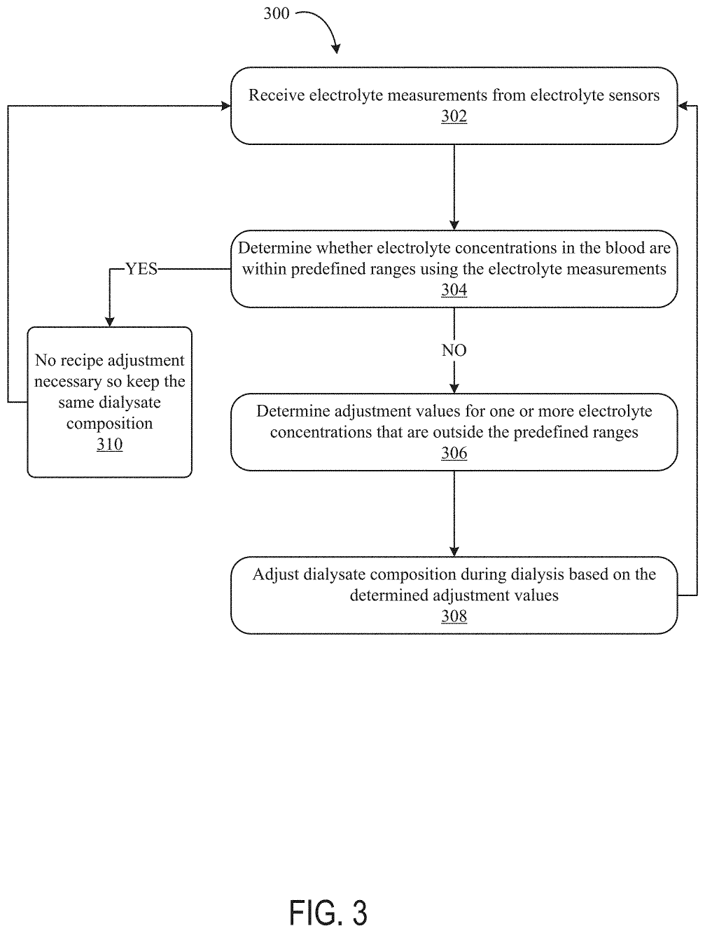

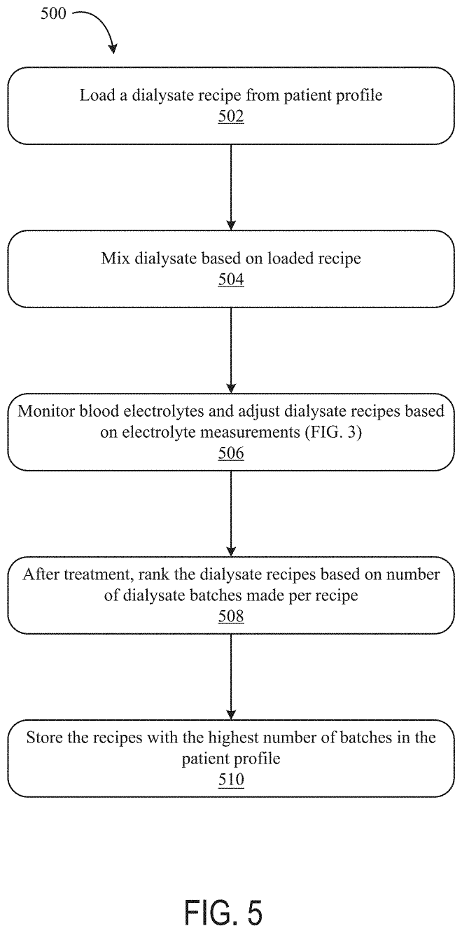

The electrolyte sensors 212 are configured to measure electrolyte concentration in the blood of the patient 210. The electrolyte sensors 212 can be, for example, conductivity sensors, nuclear magnetic resonance (NMR) sensors and/or optical sensors. NMR sensors may detect, determine, and/or obtain real-time electrolyte concentrations (e.g., sodium concentrations) in the blood of the patient 210. Additionally, and/or alternatively, NMR sensors may be modified (e.g., re-tuned to different radio frequencies) to detect, determine, and/or obtain real-time potassium and/or phosphorous concentrations. Additionally, and/or alternatively, in some embodiments, an NMR sensor measures concentrations of free sodium in both the dialysate and the blood, each sampled separately, and the concentration of free sodium is reported to the controller 101. Furthermore, the sodium and other electrolyte concentrations may vary from patient to patient and even for a given patient between consecutive dialysis sessions. Accordingly, using an NMR sensor or other sensor to measure these concentrations in real-time to adjust the electrolyte concentrations and even using individualized recipes (described in FIGS. 3 and 5) may be beneficial to provide the optimal treatment for the patient during dialysis. Additionally, and/or alternatively, the electrolyte sensors 212 may include optical sensors configured to detect real-time electrolyte concentrations such as calcium concentrations and or magnesium concentrations.

Examples of an NMR sensor usable with exemplary embodiments of the present application are described in further detail in U.S. Pat. No. 10,371,775 (Titled: Dialysis System With Radio Frequency Device Within A Magnet Assembly For Medical Fluid Sensing And Concentration Determination), granted on Aug. 6, 2019, and U.S. Provisional Patent Application No. 62/967,349 (Titled: Individualized And On-Demand Dialysis System With Networking Capabilities), filed on Jan. 29, 2019, both of which are incorporated by reference herein in its entirety.

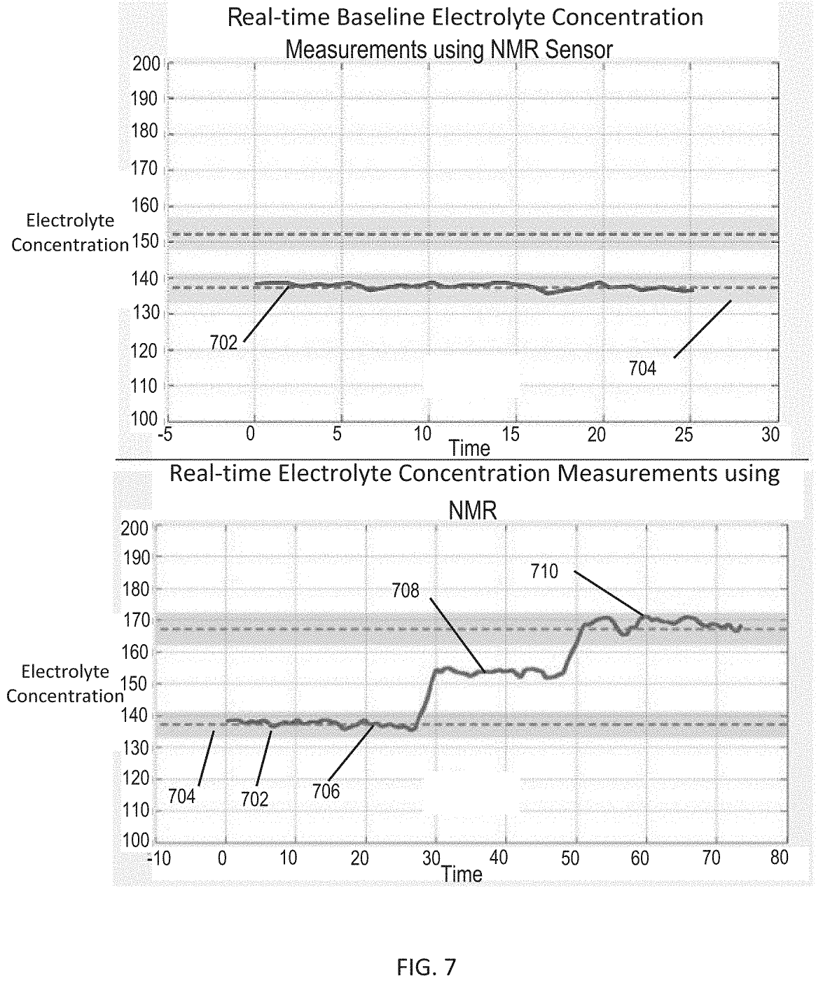

Furthermore, FIG. 7 shows graphical representations of real-time measurements obtained using an NMR sensor. For example, the top part of FIG. 7 shows real-time electrolyte concentration (e.g., sodium concentration) measurements using the NMR sensor. Line 702 indicates the sodium concentration and the shaded area 704 represents the accuracy margin. The bottom part of FIG. 7 also shows real-time electrolyte concentration measurements using the NMR sensor. For example, portion 704 of line 702 indicates the baseline sodium concentration. Then, portion 708 indicates a first adjustment of the baseline sodium concentration (e.g., introducing or injecting sodium boluses to increase the sodium concentration). Portion 710 shows another injection of sodium boluses for increasing the sodium concentration again.

FIG. 3 is a flow diagram for managing electrolytes in blood of a dialysis patient during dialysis, according to an embodiment of the disclosure. FIG. 3 is a flow diagram illustrating a process 300 that an electrolyte composition monitor, e.g., electrolyte composition monitor 200, can perform in managing electrolytes in blood of patient 210. At 302, the controller 101 of the electrolyte composition monitor 200 receives (e.g., obtains) electrolyte measurements from electrolyte sensors 212. The obtained electrolyte measurements may include sodium, potassium, phosphorous, magnesium, and/or calcium electrolyte concentrations in the blood of the patient 210.

At 304, the controller 101 determines from the electrolyte measurements whether electrolyte concentrations in the blood are within predefined ranges. In an example, electrolyte concentration of sodium in the blood should be within 135-145 mEq/L range, electrolyte concentration of potassium should be within 3.5-5 mEq/L range, electrolyte concentration of calcium should be within 8.5-10.2 mg/dL (2-2.6 mmol/L) range, and so on. The electrolyte measurements received at the controller 101 are prepared in a manner to obtain electrolyte concentrations. For example, if a sodium NMR sensor provides radio frequency (RF) energy level at a resonant frequency of sodium as measurement signals, then the controller 101 analyzes the RF energy level provided to determine the concentration of sodium in the blood. This concentration of sodium is compared to the upper and lower bounds of the predefined range for sodium to determine whether sodium concentration in the blood is within the predefined range.

In some examples, the predefined ranges are clinically defined ranges such as clinically known ranges. In other examples, the predefined ranges may be individualized. For example, as described below in 502 of FIG. 5, the dialysate recipe is a recipe determined based on historical trend analysis on electrolyte measurements from the patient's previous dialysis treatments. In other words, the dialysate recipe is individualized for the patient 210 based on the previous dialysis treatments performed on the patient 210. The dialysate recipe associated with the patient may include electrolyte ranges (e.g., an electrolyte concentration range for sodium, potassium, calcium, magnesium, and/or phosphorous). Further, as described below in FIG. 5, the controller 101 may load the dialysate recipe prior to beginning the dialysis treatment. At 302, the controller 101 determines the predefined ranges based on the loaded dialysate recipe and may compare these predefined ranges with the electrolyte concentrations from the electrolyte measurements.

At 306, the controller 101 determines adjustment values, based on the plurality of electrolyte measurements, for one or more electrolyte concentrations outside the predefined ranges. The controller 101 determines, for each electrolyte concentration outside of the predefined ranges, whether to increase or decrease concentration of the electrolyte. Increasing or decreasing the concentration provides directionality to the adjustment values. The controller 101 then determines the magnitude of the adjustment value by determining a target amount by which the concentration of the electrolyte should be increased.

In an embodiment, the controller 101 determines adjustment values by unit increments. That is, after determining whether to increase or decrease a concentration of an electrolyte that is not within a predefined range, the controller 101 determines that the concentration of the electrolyte should be adjusted by a given unit. In an embodiment where chemical constituents of dialysate are adjusted by tablets, each unit represents an electrolyte concentration provided by a chemical concentrate's respective tablet. In an embodiment where chemical constituents of dialysate are adjusted by liquid concentrates, each unit represents an expected electrolyte concentration provided by opening its respective valve for a predetermined amount of time. Although one unit increments are described, adjustment values can be determined as multiple unit increments. For example, the controller 101 can determine that the concentration of the electrolyte that is not within its predefined range should be increased by three units which correspond to an amount of electrolytes expected from three tablets.

In an embodiment, the controller 101 determines adjustment values based on pre-programmed dialysate recipes, formulas or prescriptions. The controller 101 can store one or more recipes for various electrolyte conditions in its memory. For example, the memory may include a recipe for low sodium, high sodium, low potassium, high potassium, and so on. Each of these dialysate recipes can be tagged as being effective in reducing or raising one or more electrolyte concentrations. That way, based on a combination of electrolytes determined to be outside their respective predefined ranges, the controller 101 can select a recipe from one of these predefined recipes for the next batch of dialysate.

In sonic examples and referring to FIG. 5 and process 500 below, the controller 101 determines the magnitude and/or directionality of the adjustment values (e.g., unit increments) based on the loaded dialysate recipe from 502. For example, the controller 101 may determine and load the dialysate recipe based on historical trend analysis on electrolyte measurements from the patient's previous dialysis treatments (e.g., based on the most effective recipe from the historical trend analysis, the dialysate recipe with the greatest number of batches in the patient profile, and/or the most recent dialysate recipe used during the dialysis treatment). For instance, if the high sodium recipe has the greatest number of made batches in the patient profile (e.g., the dialysate solution was created/adjusted the greatest number of times using the recipe), the controller 101 may determine the high sodium recipe as the most effective dialysate recipe and load that recipe at 502. Then, at 306, the controller 101 may determine the magnitude of the adjustment values using this recipe.

In an embodiment, the controller 101 determines that one or more electrolyte concentrations outside the predefined ranges deviates significantly from the predefined ranges. For example, at 304, a potassium concentration of 6.0 mEq/L is determined, and the predefined range for potassium is between 3.5 and 5.0 mEq/L. The potassium concentration is then determined by the controller 101 to be too high. The controller 101 can determine that the next dialysate batch should decrease the potassium concentration. Thus, the controller 101 can determine an adjustment value for potassium that reduces the potassium ion concentration in the next batch of dialysate as prescribed. Although potassium is used as an example, the controller 101 can determine that concentration of more than one electrolyte in the blood is too high and determine adjustment values to make a next batch of dialysate. In other words, the controller 101 may determine that an electrolyte concentration (e.g., potassium) is outside of the predefined ranges and determine one or more adjustment values for the next dialysate batch. The one or more adjustment values may be a single adjustment value for the electrolyte concentration (e.g., potassium) or may include multiple adjustments values for multiple different electrolyte concentrations (e.g., potassium, calcium, and so on),

Additionally, and/or alternatively, the controller 101 may determine multiple electrolyte concentrations (e.g., potassium and calcium) are outside of the predefined ranges and may determine one or more adjustment values for the next dialysate batch. The one or more adjustment values may be a single adjustment value for the electrolyte concentration (e.g., potassium) or may include multiple adjustments values for multiple different electrolyte concentrations (e.g., calcium, potassium, and so on).

In an embodiment, the controller 101 determines that a majority or all of the electrolyte concentrations are outside the predefined ranges and adjustment values of all the electrolyte concentrations have a same direction. The controller 101 can determine adjustment values based on the amounts of chemicals supplied by the chemical sources 206 and the amount of water to include in the dialysate, In some instances, each batch of the dialysate may be 12 L. In other instances, the batches may be ;greater than 12 L such as 24 L. The controller 101 can determine adjustment values based on the amount of chemicals supplied by the chemical sources 206, the amount of water to include in the dialysate, and the volume of the batch of the dialysate. For instance, if the prescription indicates that 2 potassium tablets are used for a 12 L batch, the controller 101 may determine to use 4 potassium tablets for a 24 L batch.

At 308, the controller 101 provides instructions to the dispenser 202 to adjust the composition of the dialysate during dialysis based on the determined adjustment values of 306. The composition of the dialysate includes chemicals from the chemical sources 206. In an embodiment, the controller 101 generates adjustment signals for changing the composition of the dialysate during dialysis based on the determined adjustment values. The controller 101 then provides actuating signals to actuators 208 for changing how much of each chemical concentrate to release into the mixing chamber 204. By effecting a change in an amount of any of the chemical concentrates released into the mixing chamber 204, the controller 101 causes the dispenser 202 to change proportions of the chemicals in the dialysate.

In an embodiment, when a respective adjustment value for an electrolyte concentration outside the respective predefined range indicates an increase, the controller 101 generates a respective adjustment signal for dispensing a higher proportion of a respective chemical, thus increasing a chemical contribution of a respective chemical source in the chemical sources 206. When the respective adjustment value for the electrolyte concentration outside the respective predefined range is a decrease, the controller 101 generates a respective adjustment signal for dispensing a lower proportion of the respective chemical of the respective chemical source in the chemical sources 206.

In an embodiment, the adjustment signals the controller 101 provides to the dispenser 202 are encoded as a number of electrical pulses. Electrical pulses can be voltage or current pulses. For example, a number of pulses provided by the controller 101 to a respective actuator in the actuators 208 can encode an amount of a respective chemical in the chemical sources 206 to release into the mixing chamber 204. In a previous dialysate batch, if 5 pulses were provided to an actuator that controls a release of CaCl₂ tablets into the mixing chamber 204 then for a next dialysate batch, if 4 pulses are provided to the actuator then a lower number of CaCl₂ tablets will be released into the mixing chamber 204. Thus, the adjustment signals generated by the controller 101 can be encoded as a change in a number of electrical pulses provided to one or more actuators. The change in number of electrical pulses can be an increase in the number of electrical pulses or a decrease in the number of electrical pulses. Furthermore, all but one actuator in the actuators 208 can receive a reduced number of electrical pulses. Conversely, all but one actuator in the actuators 208 can receive an increased number of electrical pulses.

After completing 308, the controller 101 cycles back to 302 and receives new electrolyte measurements (e.g., second electrolyte measurements from the sensors 212). The process 300 is performed by the electrolyte composition monitor 200 until the dialysis treatment of patient 210 ends.

For example, in subsequent iterations, at 304, the controller 101 determines from the electrolyte measurements whether the first adjustment values caused the new electrolyte measurements to be within the predefined ranges. If no electrolyte concentrations in the blood are outside the predefined ranges, then the controller 101 determines at 310 that no adjustment is necessary. The controller 101 keeps the most recent recipe for the new dialysate hatch and the process 300 returns to 302. If there are electrolyte concentrations that are still outside of the predefined ranges, the controller 101 may determine new adjustment values based on the recipe and provide additional instructions to adjust the composition of the dialysate during dialysis. Furthermore, the controller 101 may determine the effectiveness of the previous recipe used and/or determine a new recipe to use for the adjustment values.

For example, in some instances, the controller 101 may determine the effectiveness of the recipe using process 300. As described above, the controller 101 may determine the directionality and/or magnitude of the adjustment values based on the loaded recipe. For example, the controller 101 may obtain a first and a second electrolyte measurement from the electrolyte sensors 212. The first electrolyte measurement may be obtained in the first iteration of process 300 and the second electrolyte measurement may be obtained in the second iteration of process 300 (e.g., the second electrolyte measurement may be subsequent to adjusting the composition of the dialysate during dialysis). The controller 101 may compare the first electrolyte measurement, the second electrolyte measurements, and/or the predefined ranges to determine the effectiveness of the recipe. For instance, if the electrolyte concentration is within the predefined ranges after the adjustment, the controller 101 may determine the recipe used for the adjustment values at 306 is effective. If the electrolyte concentration is still not within the predefined ranges, the controller 101 may determine the recipe is not effective.

Additionally, and/or alternatively, the controller 101 may determine the effectiveness of the recipe based on how close the second electrolyte measurement is to the predefined range. For instance, if the second electrolyte measurement is within the predefined range, the controller 101 may determine the recipe is very effective. If the second electrolyte measurement is close to the predefined range, but is not within the predefined range it, the controller 101 may determine the recipe is effective. If the second electrolyte measurement is not close to the predefined range, the controller 101 may determine the recipe is not effective. If the second electrolyte measurement is even further away from the predefined range compared to the first electrolyte measurement, the controller 101 may determine the recipe is extremely ineffective.

In some variations, the controller 101 may dynamically rank recipes during the dialysis treatment (e.g., during process 300). For example, after creating each batch of the dialysate solution using the recipe, the controller 101 may determine the effectiveness of the recipe. Then, the controller 101 may determine whether to load a new dialysate recipe based on the updated effectiveness of the recipe. If the controller 101 loads a new dialysate recipe, the controller 101 may use the new dialysate recipe to determine the adjustment values. In other words, during the dialysis treatment, the controller 101 may use multiple different recipes to determine the adjustment values based on the determined effectiveness of the recipes during the treatment of the patient.

In some instances, the controller 101 may rank the recipes after the dialysis treatment for the patient has concluded (e.g., after process 300 has concluded). For example, the controller 101 may determine the effectiveness of the one or more recipes used during the dialysis treatment based on comparing the electrolyte concentration after the adjustment with the predefined ranges. Then, the controller 101 may store the associated effectiveness of the recipes in memory and/or rank the recipes based on the effectiveness. The next time the patient undergoes dialysis treatment, the controller 101 may load the highest ranking stored recipe for the predefined ranges and/or the adjustment values.

In some examples, process 300 may he used for peritoneal dialysis (PD solutions). For peritoneal dialysis, process may further include a sterilization step. For example, prior to 308, the controller 101 may provide instructions to the dispenser 202 to sterilize the composition of the dialysate including the chemicals from the chemical sources 206. Then, at 308, the controller 101 provides instructions to the dispenser 202 to sterilize the chemicals from the chemical sources.

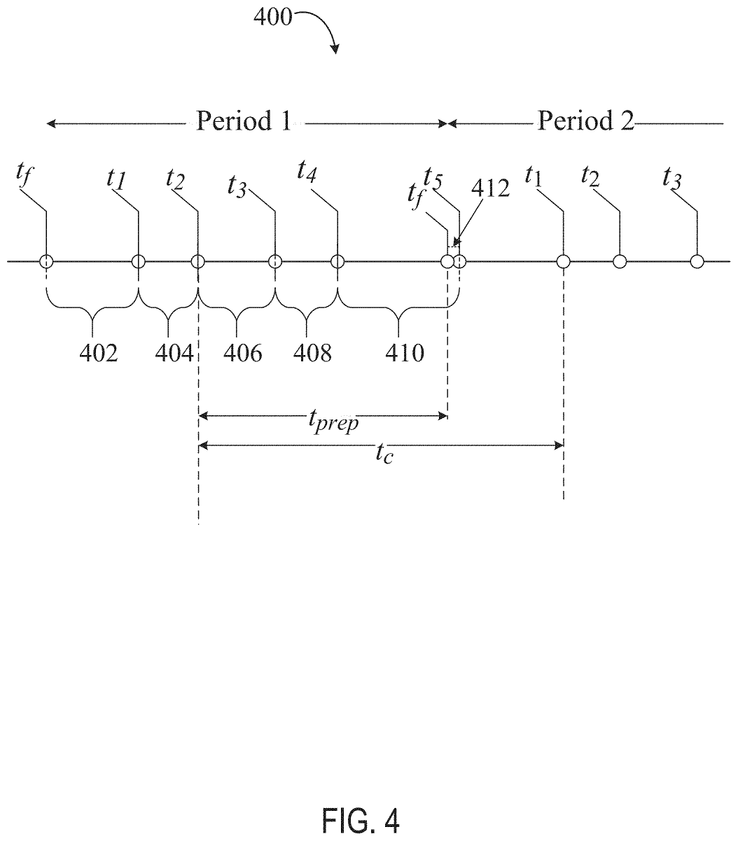

FIG. 4 illustrates an example timeline 400 for managing electrolytes in blood of a dialysis patient during dialysis. As described above with respect to FIG. 3, process 300 is cyclic or periodic, so with respect to the timeline 400, one period of activities is highlighted via timestamps t_{f}, t₁, t₂, t₃, t₄, and t₅. The timestamps are defined as follows:
t_{f}—Time when the controller 101 receives a fresh dialysate signal indicating that a new batch of dialysate is mixed and ready for use
t₁—Time when the controller 101 receives electrolyte measurements from the electrolyte sensors 212
t₂—Time when the controller 101 sends adjustment signals to the actuators 208 of the dispenser 202
t₃—Time when the actuators 208 allow chemicals and water to migrate from the chemical sources 206 and water source 205, respectively, to the mixing chamber 204
t₄—Time when the mixing chamber 204 starts mixing the new batch of dialysate
t₅—Time when an old batch of dialysate is depleted

FIG. 4 organizes activities in FIG. 3 according to the example timeline 400. In Period 1, at the start of dialysis, a fresh batch of dialysate is mixed and ready for use. At this point, the mixing chamber 204 provides a fresh dialysate signal to the controller 101 at timestamp t_{f}. After a time duration 402, the controller 101 receives, at timestamp t₁, electrolyte measurements from the electrolyte sensors 212. During a time duration 404, the controller 101 determines adjustment signals to provide to the actuators 208, and at timestamp t₂, sends the adjustment signals to the actuators 208. The actuators 208 respond to the adjustment signals after a time duration 406, so at timestamp t₃, the actuators 208 allow chemicals and water to migrate from their respective sources into the mixing chamber 204. After a time duration 408, the mixing chamber 204 then mixes its contents, at timestamp t₄, to form a new batch of dialysate.

At timestamp t₅, the old batch of dialysate is completely depleted from the mixing chamber 204, so time duration 410 indicates a time between when the mixing chamber 204 begins mixing contents for the new batch of dialysate and when the old batch of dialysate is depleted. In some embodiments, an error is not generated by the controller 101 when the new batch of dialysate is ready before the old batch of dialysate is depleted. This condition is indicated in FIG. 4 by showing that a fresh dialysate signal is provided at timestamp t_{f}during time duration 410.

In an embodiment, the controller 101 can optimize the process 300 by trying to reduce the time duration 412 between timestamps t_{f} and t₅. That way, the new batch of dialysate is ready at a same time that the old batch of dialysate is depleted so that when the fresh dialysate signal is received at the controller 101, the controller 101 can determine an appropriate time duration 402 to wait before obtaining electrolyte measurements from the electrolyte sensors 212. That way, the controller 101 gives enough time to be able to view the effects of the new batch of dialysate on the electrolytes in the blood.

Put another way, the controller 101 can monitor tₚᵣₑₚ, a time duration between when the controller 101 sends adjustment signals to the actuators 208 and when the controller 101 receives the fresh dialysate signal from the mixing chamber 204. The controller 101 can try to optimize tₚᵣₑₚ such that its duration is substantially the same as the sum of durations 406, 408, and 410.

In an embodiment, the controller 101 determines that if an adjustment signal is sent at a certain time, then there would be a violation of tₚᵣₑₚ, that is, timestamp t₅ would be reached before the new batch of dialysate is mixed and ready. The controller 101 can determine in this case to delay the adjustment signal, mix a new batch of dialysate based on an old recipe, and then provide the buffered adjustment signal in a next period. This indicates that after timestamp t_{f}, there is a maximum time tₘₐₓ, that the controller 101 can wait before sending the adjustment signals to the actuators 208 at timestamp t₂. In an embodiment tₘₐₓ, can be determined to be the sum of durations 402, 404, 406, 408, and 410 minus. Since tₘₐₓ depends on timestamp t₅, in some embodiments, tₘₐₓ, is determined by the controller 101 based on flow rate of dialysate exiting the mixing chamber 204 and a volume of dialysate in the mixing chamber 204.

In an embodiment, the controller 101 can also monitor and try to regularize t_{c}, a time duration between when the controller 101 sends an adjustment signal and when the controller 101 obtains electrolyte measurements to ascertain effects of the adjustment signals on electrolyte concentration in the blood.

FIG. 5 is a flow diagram for determining individualized dialysate recipes or prescriptions for a patient, according to an embodiment of the disclosure. FIG. 5 is a flow diagram illustrating a process 500 performed by a dialysis system, e.g., the hemodialysis system 100, to determine the patient's dialysate recipes. At 502, the hemodialysis system 100 loads a dialysate recipe from a patient profile.

In an embodiment, the hemodialysis system 100 may receive a chip card or a computer memory storage like a flash drive that contains dialysate recipes for the patient 210. In an embodiment, the patient profile may he obtained from a database or centralized storage. By way of example, for a description of a system for securely distributing information, including medical prescriptions, within a connected health network, reference is made to US Pub. No. 2018/0316505A1 to Cohen et al., which is incorporated herein by reference.

The dialysate recipe for treatment is selected and loaded from the patient profile. In an embodiment, the dialysate recipe selected is a last recipe used from a previous treatment that the patient 210 went through. In another example, the dialysate recipe selected is a default recipe especially when the patient 210 has never undergone dialysis at the specific location. In another example, the dialysate recipe selected is a recipe determined based on trend analysis of previous dialysate recipes from the patient profile. In another example, the dialysate recipe selected is a recipe determined based on historical trend analysis on electrolyte measurements from the patient's previous dialysis treatments.

At 504, the hemodialysis system 100, via the dialysate mixing system 105, mixes a first batch of dialysate based on the loaded recipe from 502.

At 506, the hemodialysis system 100 via the electrolyte composition monitor 200, monitors blood electrolytes and adjusts dialysate recipes based on electrolyte measurements according to various embodiments of the disclosure. For example, the electrolyte composition monitor 200 monitors blood electrolytes and adjusts dialysate recipes as provided in the process 300. During treatment, the hemodialysis system 100 creates a folder or a collection of dialysate entries within the patient profile for the current dialysis treatment, Within the folder, the hemodialysis system 100 can store one or more of dialysate recipe used, number of batches mixed that correspond to the dialysate recipe, and electrolyte measurements that led the dialysate recipe.

At 508, after the dialysis treatment is completed, the hemodialysis system 100 ranks the dialysate recipes stored at 506. In an embodiment, the dialysate recipes are ranked based on a number of dialysate batches made per recipe. In other words, if the hemodialysis system 100 determines the dialysate batches are effective (e.g., effective in reducing the electrolyte concentration(s) to the predefined range in 304), the hemodialysis system 100 may use the recipe again, which would increase the number of dialysate batches made using the recipe and would cause the hemodialysis system 100 to rank the dialysate recipe higher. In an embodiment, the dialysate recipes are ranked based on a trend analysis that compares similar dialysate recipes, then combines the number of batches for the similar dialysate recipes, and then ranks groups of dialysate recipes based on the combined number of batches.

In an embodiment, the similar dialysate recipes with the highest combined number of batches are analyzed to determine one representative recipe. The representative recipe can be determined via one or more statistical means, e.g., can be determined using an average, a median, a random selection, and so on.

At 510, the hemodialysis system 100 stores the dialysate recipes with the highest number of batches in the patient profile. In an embodiment, a representative recipe determined according to embodiments of the disclosure is stored along with the dialysate recipes.

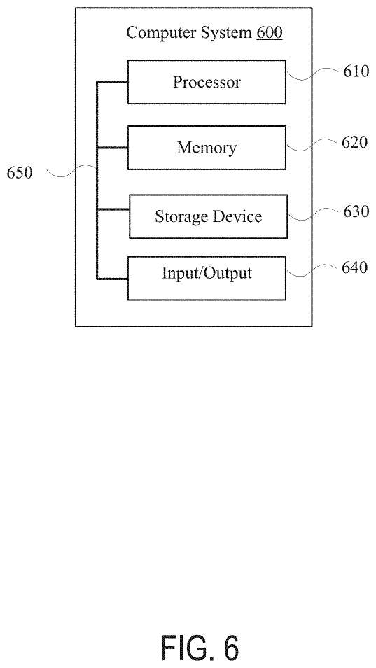

FIG. 6 is a block diagram of an example computer system 600. For example, the controller 101 is an example of the system 600 described here. The system 600 includes a processor 610, a memory 620, a storage device 630, and an input/output device 640. Each of the components 610, 620, 630, and 640 can be interconnected, for example, using a system bus 650. The processor 610 processes instructions for execution within the system 600. The processor 610 can be a single-threaded processor, a multi-threaded processor, or a quantum computer. The processor 610 can process instructions stored in the memory^{,} 620 or on the storage device 630. The processor 610 may execute operations that facilitate performing functions attributed to the electrolyte composition monitor 200.

The memory 620 stores information within the system 600. In some implementations, the memory 620 is a computer-readable medium. The mammy 620 can, for example, be a volatile memory like synchronous random access memory (SRAM) or a non-volatile memory like flash.

The storage device 630 is capable of providing mass storage for the system 600. In some implementations, the storage device 630 is a non-transitory computer-readable medium. The storage device 630 can include, for example, a hard disk device, an optical disk device, a solid-date drive, a flash drive, magnetic tape, or some other large capacity storage device. The storage device 630 may alternatively he a cloud storage device, e.g., a logical storage device including multiple physical storage devices distributed on a network and accessed using a network. In some implementations, the information stored on the memory 620 can also be stored on the storage device 630.

The input/output device 640 provides input/output operations for the system 600. In some implementations, the input/output device 640 includes one or more of network interface devices (e.g., an Ethernet card), a serial communication device (e.g., an RS-232 10 port), and/or a wireless interface device (e.g., a short-range wireless communication device, an 802.11 card, a 3G wireless modem, or a 4G wireless modem). In some implementations, the input/output device 640 includes driver devices configured to receive input data and send output data to other input/output devices, e.g., a keyboard, a printer, and display devices (such as the touch screen 118). In some implementations, the input/output device 640 receives dialysate prescription (e.g., wirelessly) for processing by the hemodialysis system 100. In some implementations, mobile computing devices, mobile communication devices, and other devices are used for sending dialysate prescriptions.

As described above, a dialysate mixing system according to embodiments of the disclosure can generate and/or make an amount of dialysate on demand using, among other things, a plurality of chemical concentrates based on a formula, recipe, or prescription. In some examples, the formula, recipe, or prescription may be generated using one or more artificial intelligence and/or machine learning (AI-ML) algorithms, datasets, or models. In some instances, the artificial intelligence (AI) algorithms may include sub-disciplines such as machine learning (ML) algorithms and/or deep learning (DL) algorithms. The AI, ML, and/or DL algorithms as well as additional and/or alternative mathematical models/modeling may be used to generate the individualized dialysis prescriptions for the patient. The generation of dialysis prescriptions will be described in further detail below.

In particular, a goal of hemodialysis is to achieve electrolyte homeostasis. However, utilizing fixed electrolyte concentrations to remove solutes, based on once-a-month measurements of serum chemistry, may be associated with and/or potentially cause un-physiologic rapid electrolyte shifts during dialysis, provoking cardiac arrhythmias and sudden cardiac death, which is the leading cause of death among dialysis patients. The ability to individualize and continuously tailor the dialysate composition in response to real-time changes in serum electrolyte levels during treatment is needed to address the hazards arising from the rapid intradialytic electrolyte shifts.

There are currently nephrologist/medical staff shortages worldwide, which makes it difficult for a patient to obtain their own individualized dialysis prescription. This is especially true as the renal disease patient population worldwide is increasing, which is also causing nephrologists to spend less time with their patients. Furthermore, even if patients were treated with their own individualized dialysis prescription, there are currently few tools, if any, to analyze how the individualized dialysis prescriptions are impacting the patient's outcome. Additionally, there is a vast amount of previous treatment information and outcomes for dialysis patients available, but there is no implemented mechanism to use this vast amount of data in gaining the insight to generate new dialysis prescriptions. Today's dialysis treatments have largely remained unchanged over the past decades. The present disclosure describes technology tools and methods such as using AI-ML algorithms and/or models to generate individualized dialysis prescriptions. By using AI-ML algorithms, more accurate individualized dialysis prescriptions may be generated and provided to treat patients, which may in turn better prevent cardiac arrhythmias, sudden cardiac death, and/or other medical complications. In addition to the improved patient benefits and greater accuracy, the individualized dialysis prescription may be generated and controlled in real time in an automated manner using AI-ML algorithms, which may help with the growing shortage of nephrologists as well as the increase in patients with renal diseases.

As described herein, a dialysis system (e.g., the hemodialysis system 100 that includes the hemodialysis machine 102) is designed to generate completely customizable dialysate prescriptions that can be individualized for each treatment and/or patient. Furthermore, this prescription can be continuously adjusted throughout treatment. In other words, the dialysis system, married with on-line point-of-care serum electrolyte assessments, may allow for precise control of electrolyte levels during treatment, potentially reducing mortality due to inadequate electrolyte homeostasis. Additionally, the ability to generate small batches of dialysate solution on demand helps solve an important and common barrier to home hemodialysis, the necessity of delivering and storing large volumes of concentrates used for preparing the dialysis prescription.

The dialysis system also aims to achieve an appropriate fluid balance to allow normalization of blood-water volumes and alleviate symptoms of fluid overload (swelling, hypertension, pulmonary edema). Sodium, the major determinant of extracellular fluid volume, obtained via dietary sources or dialysate, can significantly alter fluid balance. Excess sodium intake from dialysate may lead to fluid overload and even hospitalization or death. Therefore, clinicians try to limit sodium concentration. However, higher dialysate sodium also improves symptoms—hypotension and/or muscle cramps. The clinician may need to make a difficult choice of balancing excess sodium delivery with symptom relief when determining dialysate sodium prescriptions. Complicating the problem, pre-dialysis plasma sodium levels vary from patient-to-patient and even from treatment-to-treatment in the same patient, For improved volume regulation, an ideal dialysis machine would have the capacity to measure plasma sodium and adjust dialysate-to-serum concentration gradients within a single dialysis session. This, combined with effective fluid removal by controlled and safe ultrafiltration (UF) rate may allow major enhancements in personalizing volume regulation at the patient and treatment level. Furthermore, patients should be provided with tools to view and manage the volume status, implied by real-time sodium values in plasma and prescribed dialysate.

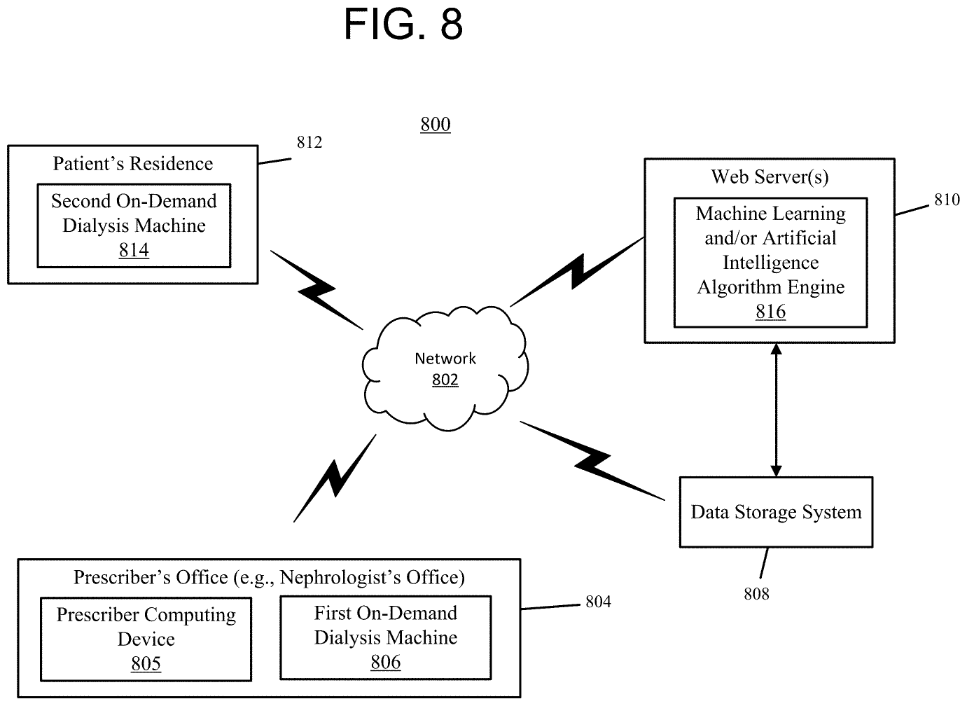

FIG. 8 is a simplified block diagram depicting an exemplary individualized and on-demand dialysis system 800 with network capabilities. The system 800 includes a prescriber's office 804, a network 802, a data storage system 808, an enterprise web server 810, and a patient's residence 812. The entities and devices within system 800 may be connected to and/or communicate over a network 802, The network 802 may be a global area network (GAN) such as the Internet, a wide area network (WAN), a local area network (LAN), or any other type of network or combination of networks. The network 802 may provide a wireline, wireless, or a. combination of wireline and wireless communication between the entities within the system 800. In some instances, the web server 810 may be in communication with the data storage system 808 without using the network 802. For instance, the web server 810 may use one or more communication protocols such as WI-FI or BLUETOOTH and/or a wired connection to communicate with the data storage system 808. In some examples, the data storage system 808 may be included within the web server 810.

In operation, the prescriber's office (e.g., nephrologist's office) 804 may use the network 802 to communicate with one or more devices, residences, and/or other systems within the system 800. In some examples, a prescriber such as a nephrologist may prescribe a dialysis prescription for a patient. The dialysis prescription may be individualized. For example, the prescription may indicate different levels of electrolytes such as sodium, potassium, magnesium, calcium, and/or other prescriptions/parameters for the particular patient.

The prescriber may use a computing device (e.g., computing device 805) to input the information indicating the dialysis prescription. For instance, the prescriber's office may include a prescriber computing device 805. The computing device 805 may include one or more processors, memory, and/or additional components to communicate with one or more devices within the system 800. In some examples, after inputting the prescription, the prescriber computing device 805 may provide information to the data storage system 808.

The data storage system 808 may be any system or set of systems that stores information and/or communicates with one or more devices within system 800. For example, the data storage system 808 may store the individualized prescriptions for one or more patients after receiving the information from the prescriber computing device 805 and/or other computing devices including computing devices from other prescribers/nephrologists. The data storage system 808 may also store past patient information, prescriptions given, and/or may receive information tracking the prescription/patient outcomes. For example, a new patient entering dialysis for the first time may be put into an applicable cluster (e.g., by using ML algorithms described below), and a prescription may be suggested by the machine learning and/or artificial intelligence (AI-ML) algorithms engine 816. The nephrologist may choose to accept, edit or reject the recommendation.

The patient's residence 812 may be a dwelling or home of the patient. The prescriber's office 804 and; or the patient's residence 812 may include an on-demand dialysis machine 806 and 814. In some instances, the on-demand dialysis machine 806 and/or 814 may be the dialysis system shown in FIG. 1 (e.g., the hemodialysis system 100 and/or the hemodialysis machine 102) and may be used to perform dialysis treatment, including hemodialysis treatment, on a patient. For example, the patient may receive treatment at the prescriber's office using the first on-demand dialysis machine 806. Additionally, and/or alternatively, the patient may receive treatment at the patient's residence 812 using the second on-demand dialysis machine 814. The treatment performed by the first and/or second on-demand dialysis machines 806 and/or 812 may be based on the individualized dialysis prescription. For example, as described above, the controller 101 of the dialysis machine 102 may compute the chemical composition for a dialysate and create the batch of dialysate for the patient based on the dialysis prescription. In some instances, the dialysis machines 806 and/or 812 may be capable of generating the ‘standard’ dialysate with fixed formulations. In some examples, the batches needed for the entire treatment duration may be made sequentially following the prescriber's program/information sent to the machine. The total number of batches to be made may depend on the duration of the dialysis treatment and dialysate flow (milliliter/minute (ml/min)). In some variations, the on-demand dialysis machines 806 and/or 814 may be peritoneal dialysis (PD) machines. In instances where the on-demand dialysis machines 806 and/or 814 are PD machines, the PD fluid may be sterilized with a specific filter at the point of care. In some examples, the on-demand dialysis machines 806 and/or 814 may include a water purification system that is used to prepare the dialysate.

The first and second on-demand dialysis machine 806 and/or 814 may communicate and receive the individualized prescriptions for the patients from one or more devices within the system 800 such as the prescriber computing device 805, and/or the data storage system 808. For example, the first and/or second on-demand dialysis machine 806, 814 may provide patient identification information to the data storage system 808. In response, the first and/or second on-demand dialysis machine 806, 814 may receive, from the data storage system 808, the individualized prescriptions. The first and/or second on-demand dialysis machine 806, 814 may use the individualized prescriptions to provide an individualized dialysis treatment for the patient. In other words, by including communication devices such as databases, servers, cloud computing platforms, and so on, the patients may receive their individualized dialysis treatments in their own residence 812 and/or in clinics such as at the prescriber's offices 804.

After the treatment has been completed, the first and/or second on-demand dialysis machine 806, 814 may provide treatment information including the treatment outcomes to the data storage system 808. For example, the data storage system 808 may store previous dialysis treatments for the patient and/or other patients.

In some instances, the prescriber may be recommended one or more dialysis treatments for the patient using an AI-ML algorithms and/or dataset. The prescriber may accept, modify, and/or decline the recommended treatment from the machine learning and/or artificial intelligence algorithm. For example, the web server 810 may be one or more servers and/or other systems within the system 800. The web server 810 may include one or more processors which execute the AI-ML algorithm engine 816.

The AI-ML algorithm engine 816 may use one or more AI-ML algorithms and/or datasets to determine and recommend an individualized dialysis prescription for the patient to a nephrologist. For instance, the dataset used to determine the individualized dialysis prescription for the patient may be utilized to develop a supervised ML algorithm (e.g., a person and/or another device instructs the engine 816 of what the AI-ML dataset should learn and provides the data to train and test the dataset). Additionally, and/or alternatively, the AI-ML dataset may be utilized to develop an unsupervised AI-ML algorithm. For unsupervised AI-ML datasets, a person and/or another device provides vast data. to the engine 816 to train and test the AI-ML algorithm, but does not instruct the engine 816 on what the algorithm and/or dataset should learn. Based on the vast data, the engine 816 may use the trained AI-ML dataset to determine and form similar patient clusters. The patient clusters may be associated with one or more individualized dialysis prescriptions.

In other words, the engine 816 may receive information (e.g., vast data) such as past prescriptions (e.g., previously prescribed individualized dialysis prescriptions), outcomes, and potential recommended prescriptions. Using this information, the engine 816 may train, test, and/or implement an AI-ML algorithm. The engine 816 may input information associated with a new or existing patient into the trained AI-ML dataset and the trained AI-ML dataset may output a patient cluster associated with the new or existing patient. For instance, the patient cluster may be a cluster or grouping of similar patients such as a cluster of hyperkalemic patients. Each of these patient clusters may be associated with one or more individualized dialysis prescriptions.

In some variations, the dataset may be used in a Reinforced AI-ML algorithm. For Reinforced AI-ML, the engine 816 may use a reward state to train the AI-ML algorithm (e.g., if the response improves the current state, then reward and if not, then penalize).

The AI-ML algorithm engine 816 may receive treatment information from the data storage system 808. This information may include, but is not limited to, past prescriptions, outcomes, and/or recommended prescriptions. Based on the received information, the AI-ML algorithm engine 816 may train the AI-ML dataset for use in determining patient clusters and/or individualized prescriptions for patients. In some instances, the AI-ML algorithm engine 816 may receive training information from one or more devices such as one or more dialysis machines (e.g., the first and/or second on-demand dialysis machines 806, 814). For example, a large chunk of data (“big data”) may be divided into training data (−70%) and test data (−30%). The engine 816 may have a specific objective (e.g., finding associations between input i.e.. patient disease conditions and output patient condition due to dialysis prescriptions). The engine 816 may use methods such as Support Vector Machine (SVM) and/or K Nearest Neighbor (kNN) to accomplish this objective. For instance, the engine 816 may use SVM and kNN to form patient clusters based on the associations or similarities of the different patients within the data.

After training the AI-ML, module, the AI-ML, algorithm engine 816 may input new data (e.g., data associated with a new or existing patient) into the trained AI-ML module to determine and/or identify a particular patient cluster and/or treatment cluster for the patient. The patient cluster or treatment cluster may be a cluster or grouping of patients with similar symptoms, medical conditions, and/or undergoing similar medical treatments. For instance, the patient/treatment cluster may be a cluster of patients with medical conditions such as common electrolyte imbalances in end-stage renal disease (ESRD) hyponatremia, hyperkalemia, and so on. The AI-ML, algorithm engine 816 may recommend individualized prescriptions for the new or existing patient using the past dataset on patient outcomes.

The AI-ML algorithm engine 816 may provide the output from the AI-ML dataset (e.g., the identified patient cluster and/or the individualized prescription) to a device such as the prescriber computing device 805, the first on-demand dialysis machine 806, and/or the second on-demand dialysis machine 814. The first and/or second on-demand dialysis machines 806, 814 may perform dialysis treatment on the patient using the individualized dialysis prescription that was determined by the AI-ML, algorithm engine 816. For instance, referring to FIGS. 5 and 502, the controller 101 may load the dialysate recipe (e.g., the individualized dialysis prescription) that was generated by the AI-ML algorithm engine 816. Then, the process 500 may proceed as described above in FIG. 5.

In some examples, the prescriber (e.g., nephrologist) may review, examine, modify, revise, accept, reject, and/or override the output (e.g., the prescription) recommended by AI-ML. For example, the prescriber computing device 805 may receive the output of the AI-ML dataset (e.g., the identified data cluster and/or the individualized prescription) and cause display of the output. The prescriber may provide user input to the prescriber computing device 805 indicating whether the output is acceptable and/or additional modifications/revisions to the output, The prescriber computing device 805 may provide the user input (e.g., whether the output from the AI-ML dataset is acceptable and/or modifications/revisions to the output) to the AI-ML algorithm engine 816. The AI-ML algorithm engine 816 may use this data to train and/or further train the AI-ML dataset.

In some instances, the engine 816 may provide an identified patient cluster or treatment cluster for the patient to the prescriber computing device 805. The prescriber, using the device 805, may provide input indicating whether the identified patient cluster or treatment cluster is acceptable. If the patient treatment cluster is acceptable, the prescriber computing device 805 may determine an individualized dialysis prescription for the patient (e.g., the prescriber may prescribe a dialysis prescription based on the patient/treatment cluster). Additionally, and/or alternatively, if the identified patient/treatment cluster is not acceptable, the prescriber may provide additional input indicating a new or modified patient/treatment cluster for the patient. The input indicating whether the patient/treatment cluster is acceptable as well as the additional input indicating a new or modified patient/treatment cluster may be provided back to the engine 816 and the engine 816 may use this input to train/further train the AI-ML dataset.

In some examples, the engine 816 may provide an individualized dialysis prescription for the patient to the prescriber computing device 805. The prescriber, using the device 805, may provide input indicating whether the determined individualized dialysis prescription is acceptable. Additionally, and/or alternatively, if the determined individualized dialysis prescription is not acceptable, the prescriber may provide additional input indicating a new or modified individualized dialysis prescription for the patient. The input indicating whether the individualized dialysis prescription is acceptable as well as the additional input indicating a new or modified individualized dialysis prescription may be provided back to the engine 816 and the engine 816 may use this input to train/further train the AI-ML dataset.

After determining the individualized dialysis prescription, the prescriber computing device 805 may provide information indicating the individualized dialysis prescription to the first and/or second on-demand dialysis machines 806 and/or 814. The first and/or second on-demand dialysis machines 806 and/or 814 may provide dialysis treatment to the patient based on the individualized dialysis prescription.

In some variations, the patient may be receiving the treatment at home (e.g., using the second on-demand dialysis machine 814). The second on-demand dialysis machine 814 may require a patient log-in prior to performing the dialysis treatment. For example, the second on-demand dialysis machine 814 may receive user input indicating a fingerprint, code (e.g., QR code), and/or other identification indicating the identity of the patient. The second on-demand dialysis machine 814 may verify or deny the patient identification. Based on verifying the patient, the second on-demand dialysis machine 814 may perform dialysis treatment on the patient using the individualized dialysis prescription.

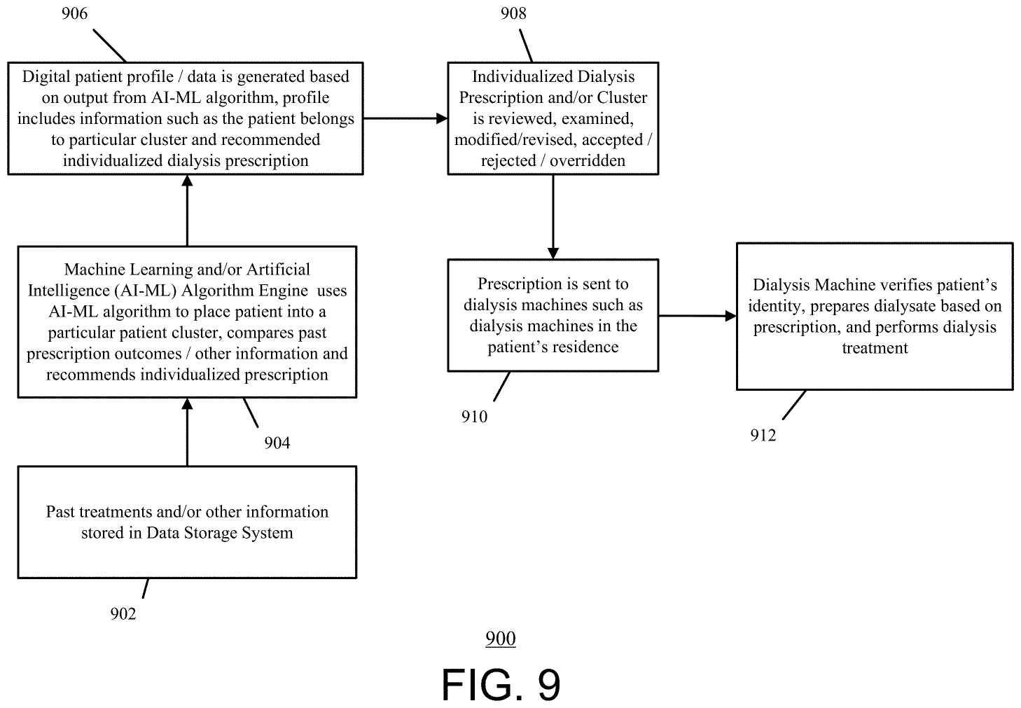

FIG. 9 illustrates an exemplary process 900 for using AI-ML to provide individualized dialysis prescriptions and treatments to patients. In operation, at block 902, the past treatments and/or other information (e.g., previously determined dialysis prescriptions and/or patient outcomes based on the prescriptions) may be stored in the data storage system 808. The AI-ML algorithm engine 816 may retrieve this information from the data storage system 808.

At block 904, the AI-ML algorithm engine 816 (e.g., an artificial intelligence (AI) module) uses one or more AI-ML algorithms to place a patient into a particular patient cluster, compares past prescription outcomes/other information, and recommends an individualized prescription.

At block 906, the AI-ML algorithm engine 816 generates a digital patient profile and/or additional data based on the output from the AI-ML algorithm. The profile includes information such as the patient belongs to a particular patient cluster and a recommended individualized dialysis prescription.

At block 908, the individualized dialysis prescription and/or cluster from the digital patient profile may be reviewed, modified/revised, accepted/rejected/overridden. For example, the AI-ML algorithm engine 816 may provide the individualized dialysis prescription/cluster to the prescriber computing device 805. The prescriber computing device 805 may display the dialysis prescription and/or cluster. Then, the nephrologist may review, modify, revise, accept, reject, and/or override the output from the AI-ML algorithm engine 816 (e.g., the recommended dialysis prescription cluster).

At block 910, the prescription is sent to one or more dialysis machines such as a dialysis machine 814 in the patient's residence 812. For instance, the prescriber computing device 805 may provide the prescription/cluster to the dialysis machines 806 and/or 814. Additionally, and/or alternatively, the prescription may first be sent to a database (e.g., the data storage system 808) and/or a cloud computing service. The database/cloud computing service may then forward this to the dialysis machine such as a home hemodialysis machine.

At block 912, the dialysis machines (e.g., 806 and/or 814) verifies the patient's identity, prepares dialysate based on the prescription, and performs the dialysis treatment. For example, after receiving the individualized dialysis prescription, the dialysis machine may prompt the patient to log-in and verify their identity. Treatment will he denied/cancelled if the patient is not verified. If the patient is verified, the dialysis machine may prompt the patient to ensure the power is on, water is connected, cassettes are loaded, other pre-treatment predations are completed, and/or it is connected to the blood circuit. Then the dialysate is prepared based on the individualized dialysis prescription. The machine may make any fluid based on the chemicals contained in the tableted form in cassettes. After the preparations are completed, the patient may undergo dialysis treatment as described above.

Although millions of hemodialysis treatments were given in the past and prior to using AI-ML, the collected data was rarely used in understanding the trends between the patient morbidities, prescribed prescriptions, and outcomes. With the advent of AI-ML, data science methods such as kNN and/or SVM may be used to define patient clusters (e.g., common electrolyte imbalances in ESRD hyponatremia, hyperkalemia). For example, a new patient entering stage 5 ESRD needing dialysis may be classified into an applicable cluster. Since past prescriptions and outcomes have been examined to understand the trend, it is possible use this information in prescribing an individualized dialysis prescription for the patient.

In some instances, the engine 816 may examine calcium (Ca) prescriptions used in various clinics. In some examples, the engine 816 may determine associations between co-morbidities (e.g., hypokalemic patient may also have hypomagnesemia) that may not be readily apparent and use the determined associations to determine individualized dialysis prescriptions.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to.”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred. embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An individualized and on-demand dialysis system, comprising:
a prescription recommendation server configured to:
receive, from a prescriber computing device, patient information associated with a new patient;
determine, based on the patient information, an individualized dialysis prescription for the new patient, wherein the individualized dialysis prescription indicates a particular patient cluster associated with the new patient; and
transmit, to an on-demand dialysis machine, the individualized dialysis prescription for the new patient; and
the on-demand dialysis machine configured to:
receive, from the prescription recommendation server, the individualized dialysis prescription for the new patient; and
perform a dialysis treatment on the new patient based on the individualized dialysis prescription.

2. The individualized and on-demand dialysis system of claim 1, wherein the prescription recommendation server is configured to determine the individualized dialysis prescription for the new patient based on using one or more dialysis prescription machine learning and/or artificial intelligence (AI-ML) models.

3. The individualized and on-demand dialysis system of claim 2, wherein the prescription recommendation server is configured to determine the individualized dialysis prescription based on using the one or more dialysis prescription AI-ML models by:
inputting the patient information into the one or more dialysis prescription AI-ML models to determine the particular patient cluster, wherein the particular patient cluster is associated with a medical condition of the new patient; and
determining the individualized dialysis prescription based on the particular patient cluster.

4. The individualized and on-demand dialysis system of claim 2, wherein the prescription recommendation server is further configured to:
train the one or more dialysis prescription AI-ML models based on received training information to determine associations within the received training information.

5. The individualized and on-demand dialysis system of claim 4, wherein the prescription recommendation server is further configured to:
receive the training information, wherein the training information comprises past prescriptions provided to a plurality of patients, outcomes associated with performing dialysis treatment using the past prescriptions, and a plurality of recommended dialysis prescriptions.

6. The individualized and on-demand dialysis system of claim 2, wherein the one or more dialysis prescription AI-ML models comprises a supervised AI-ML model, wherein the supervised AI-ML model is a support vector machine (SVM) model or a K Nearest Neighbor (kNN) model.

7. The individualized and on-demand dialysis system of claim 1, wherein the prescriber computing device and the on-demand dialysis machine are both physically located at a prescriber's office.

8. The individualized and on-demand dialysis system of claim 1, wherein the prescriber computing device is physically located at a prescriber's office associated with a first geographical location, and
wherein the on-demand dialysis machine is physically located at a residence of the new patient, wherein the residence is associated with a second geographical location that is different from the first geographical location.

9. The individualized and on-demand dialysis system of claim 1, wherein the prescription recommendation server is further configured to:
transmit, to the prescriber computing device, the individualized dialysis prescription for the new patient; and
receive, from the prescriber computing device, prescriber information indicating one or more adjustments to the individualized dialysis prescription, and
wherein the prescription recommendation server is configured to transmit the individualized dialysis prescription for the new patient by transmitting the individualized dialysis prescription with the one or more adjustments indicated by the prescriber information.

10. A method, comprising:
receiving, by a prescription recommendation server and from a prescriber computing device, patient information associated with a new patient;
determining, based on the patient information, an individualized dialysis prescription for the new patient, wherein the individualized dialysis prescription indicates a particular patient cluster associated with the new patient; and
transmitting, to an on-demand dialysis machine, the individualized dialysis prescription for the new patient, wherein the on-demand dialysis machine performs a dialysis treatment on the new patient based on the individualized dialysis prescription.

11. The method of claim 10, wherein determining the individualized dialysis prescription for the new patient is based on using one or more dialysis prescription machine learning and/or artificial intelligence (AI-ML) models.

12. The method of claim 11, wherein determining the individualized dialysis prescription based on using the one or more dialysis prescription AI-ML models comprises:
inputting the patient information into the one or more dialysis prescription AI-ML models to determine the particular patient cluster, wherein the particular patient cluster is associated with a medical condition of the new patient; and
determining the individualized dialysis prescription based on the particular patient cluster.

13. The method of claim 11, further comprising:
training the one or more dialysis prescription AI-ML models based on received training information to determine associations within the received training information.

14. The method of claim 13, further comprising:
receiving the training information, wherein the training information comprises past prescriptions provided to a plurality of patients, outcomes associated with performing dialysis treatment using the past prescriptions, and a plurality of recommended dialysis prescriptions.

15. The method of claim 11, wherein the one or more dialysis prescription AI-ML models comprises a supervised AI-ML model, wherein the supervised AI-ML model is a support vector machine (SVM) model or a K Nearest Neighbor (kNN) model.

16. The method of claim 10, wherein the prescriber computing device and the on-demand dialysis machine are both physically located at a prescriber's office.

17. The method of claim 10, wherein the prescriber computing device is physically located at a prescriber's office associated with a first geographical location, and
wherein the on-demand dialysis machine is physically located at a residence of the new patient, wherein the residence is associated with a second geographical location that is different from the first geographical location.

18. The method of claim 10, further comprising:
transmitting, to the prescriber computing device, the individualized dialysis prescription for the new patient; and
receiving, from the prescriber computing device, prescriber information indicating one or more adjustments to the individualized dialysis prescription, and
wherein transmitting the individualized dialysis prescription for the new patient comprises transmitting the individualized dialysis prescription with the one or more adjustments indicated by the prescriber information.

19. A non-transitory computer-readable medium having processor-executable instructions stored thereon, wherein the processor-executable instructions, when executed, facilitate:
receiving, from a prescriber computing device, patient information associated with a new patient;
determining, based on the patient information, an individualized dialysis prescription for the new patient, wherein the individualized dialysis prescription indicates a particular patient cluster associated with the new patient; and
transmitting, to an on-demand dialysis machine, the individualized dialysis prescription for the new patient, wherein the on-demand dialysis machine performs a dialysis treatment on the new patient based on the individualized dialysis prescription.

20. The non-transitory computer-readable medium of claim 19, wherein determining the individualized dialysis prescription for the new patient is based on using one or more dialysis prescription machine learning and/or artificial intelligence (AI-ML) models.
